(19) United States
(12) Patent Application Publication
(10) Pub. No.: US 2013/0209625 A1
(43) Pub. Date: Aug. 15, 2013

(54) METHODS AND COMPOSITIONS FOR STIMULATING A FELIDAE T1R RECEPTOR

(71) Applicants: Joseph G. Brand, Wayne, PA (US); Bruce Bryant, Elkins Park, PA (US)

(72) Inventors: Joseph G. Brand, Wayne, PA (US); Bruce Bryant, Elkins Park, PA (US)

(73) Assignees: MONELL CHEMICAL SENSES CENTER, Philadelphia, PA (US); SPECIALITES PET FOOD, Elven (FR)

(21) Appl. No.: 13/812,416

(22) PCT Filed: Jul. 11, 2011
(86) PCT No.: PCT/EP11/61786

§ 371 Date: Apr. 5, 2013

Related U.S. Application Data
(60) Provisional application No. 61/367,930, filed on Jul. 27, 2010.

(51) Int. Cl. G01N 33/50; A23K 1/175; G01N 33/68; G01N 33/84; C12N 5/0793

(52) U.S. Cl. CPC G01N 33/5041; G01N 33/84; C12N 5/062; G01N 33/68; A23K 1/1755
USPC 426/231; 506/10; 435/34; 435/375; 426/534

(57) ABSTRACT

Provided herein are methods for stimulating the T1R1/T1R3 receptor comprising contacting the receptor with pyrophosphate and one or more amino acids. Also provided, is a Felidae food product comprising pyrophosphate and one or more amino acids. Methods for determining the pyrophosphate-augmented activity of a test compound on the T1R1/T1R3 receptor are also described, as well as methods for adjusting the food formulation for Felidae.

### TECHNICAL FIELD

The present invention relates generally to the field of taste receptor stimulation. Specifically, the present invention relates to the discovery of methods of stimulating the Felidae T1R1/T1R3 receptor by contacting the receptor with pyrophosphate or with pyrophosphate and one or more amino acids. The present invention also relates to Felidae food products comprising pyrophosphate and one or more amino acids.

### BACKGROUND

The T1R class of taste receptors normally comprises three members: T1R1, T1R2, and T1R3. The dimer T1R2/T1R3 responds to sweet stimuli, while the dimer of T1R1/T1R3 recognizes amino acids (umami sensation in humans).

The cat and its relatives within the Felidae family possess the gene for T1R2, Tas1r2, but with a micro-deletion encompassing 247 by (basepairs) within exon 3. The 247 by deletion causes a frameshift that generates premature stop codons in exons 4 and 6. No protein product of the Felidae Tas1r2 gene can be located, making it a likely “pseudogene.”

Loss of a functional Felidae T1R2 receptor implies loss of a positive dimension of flavor in cats. Novel methods and compositions for specifically and efficiently stimulating the Felidae T1R1/T1R3, which thus appears to be the sole functional T1R class receptor in felidae are highly desirable.

### SUMMARY

Provided herein are methods for determining if two or more test compounds have an augmenting effect on the T1R1/T1R3 receptor where each test compound alone elicits a small or moderate response comprising contacting the receptor with the test compounds, and measuring the response of the receptor to the test compounds, wherein the compounds have an augmenting effect on the receptor if the response of the receptor to the compounds in a mixture in which each compound is presented at a given concentration is greater than the response to either one of the compounds alone at its given respective concentration. Responses are measured in terms of the Fluorescence Ratio F₃₄₀/F₃₈₀ as detailed in the Examples. Responses are gauged by comparing each response with the response to 100 μM isoproterenol, the positive control which activates the cell through Gα15. A null response is less than 10% of the control response. A small or moderate response is equal to or greater than 10% of the control response. In some preferred embodiments, the T1R1/T1R3 receptor is a Felidae T1R1/T1R3 receptor

In preferred embodiments of the methods for determining if two or more test compounds have an augmenting effect on the T1R1/T1R3 receptor, the response of the receptor to the test compound is measured by calcium imaging. In preferred embodiments, the calcium imaging is conducted with a fluorescent dye, including but not limited to acetoxymethyl esters, for example, Calcium Green 1-AM™, Fura2-AM™, and Fura Red AM™.

In preferred embodiments of the methods for determining if two or more test compounds have an augmenting effect on the T1R1/T1R3 receptor, a cell-based assay is employed to measure the response of the receptor to the test compound. In further preferred embodiments, the cell-based assay comprises HEK293-Gα15 cells that express one or more polynucleotides encoding a Felidae T1R1/T1R3 receptor. In yet further preferred embodiments, the cell-based assay comprises HEK293-Gα15 cells that are transfected with one or more polynucleotides encoding a Felidae T1R1/T1R3 receptor.

In preferred embodiments of the methods for determining if two or more test compounds have an augmenting effect on the T1R1/T1R3 receptor, one or more test compounds is an amino acid. In some preferred embodiments, one test compound is pyrophosphate. In some preferred embodiments, the one or more amino acids is present at a final concentration equal to or greater than 1 mM and/or the pyrophosphate is present at a final concentration equal to or greater than 1 mM. In some preferred embodiments, the one or more amino acids is present at a final concentration equal to or greater than 1 mM and/or the pyrophosphate is present at a final concentration equal to or greater than 5 mM. In some preferred embodiments, the one or more amino acids is present at a final concentration equal to or greater than 1 mM and/or the pyrophosphate is present at a final concentration equal to or greater than 10 mM. In some preferred embodiments, the one or more amino acids is present at a final concentration equal to or greater than 1 mM and/or the pyrophosphate is present at a final concentration equal to or greater than 20 mM. In further preferred embodiments, the one or more amino acids is present at a final concentration equal to or greater than 1 mM and/or the pyrophosphate is present at a final concentration equal to or greater than 25 mM. In yet further preferred embodiments, the one or more amino acids is present at a final concentration equal to or greater than 1 mM and/or the pyrophosphate is present at a final concentration equal to or greater than 30 mM. In some preferred embodiments, the one or more amino acids is present at a final concentration equal to or greater than 50 mM and/or the pyrophosphate is present at a final concentration equal to or greater than 1 mM. In some preferred embodiments, the one or more amino acids is present at a final concentration equal to or greater than 50 mM and/or the pyrophosphate is present at a final concentration equal to or greater than 5 mM. In some preferred embodiments, the one or more amino acids is present at a final concentration equal to or greater than 50 mM and/or the pyrophosphate is present at a final concentration equal to or greater than 10 mM. In some preferred embodiments, the one or more amino acids is present at a final concentration equal to or greater than 50 mM and/or the pyrophosphate is present at a final concentration equal to or greater than 20 mM. In further preferred embodiments, the one or more amino acids is present at a final concentration equal to or greater than 50 mM and/or the pyrophosphate is present at a final concentration equal to or greater than 25 mM. In yet further preferred embodiments, the one or more amino acids is present at a final concentration equal to or greater than 50 mM and/or the pyrophosphate is present at a final concentration equal to or greater than 30 mM. In preferred embodiments, the amino acid is pro line present at a final concentration of 25 mM and/or the pyrophosphate is present at a final concentration of 20 mM. In further preferred embodiments, the amino acid is pro line present at a final concentration of 50 mM and/or the pyrophosphate is present at a final concentration of 20 mM. In yet further preferred embodiments, the amino acid is lysine present at a final concentration of 25 mM and/or the pyrophosphate is present at a final concentration of 20 mM. In further preferred embodiments, the amino acid is alanine present at a final concentration of 25 mM and/or the pyrophosphate is present at a final concentration of 20 mM. In yet further preferred embodiments, the amino acid is histidine present at a final concentration of 25 mM and/or the pyrophosphate is present at a final concentration of 20 mM. In preferred embodiments, the pyrophosphate is sodium pyrophosphate. In further preferred embodiments, the pyrophosphate is trisodium pyrophosphate, or Na₃HP₂O₇. In some embodiments, “final concentration” means the concentration of the compound in the tasting solution superfused in the recording chamber of the calcium imaging technique.

Provided herein are methods for selecting a cell line that is suitable for carrying out the methods for determining if two or more test compounds have an augmenting effect on the T1R1/T1R3 receptor. The selection methods comprise contacting one or more cell lines with isoproterenol, and conducting calcium imaging to measure the response of the endogenous isoproterenol receptor in the cell line, and selecting a cell line that exhibits a response by the endogenous isoproterenol receptor.

Also provided is a method for generating a cell line expressing the T1R1/T1R3 receptor comprising contacting a cell line with isoproterenol, conducting calcium imaging to measure the response of the endogenous isoproterenol receptor in said cell line, selecting a cell line that responds to isoproterenol and transfecting the selected cell line with one or more polynucleotides encoding the T1R1/T1R3 receptor.

Provided is a method for determining the pyrophosphate-augmented activity of a test compound on the T1R1/T1R3 receptor if each test compound alone elicits a small or moderate response comprising contacting one or more cells expressing one or more polynucleotides encoding the T1R1/T1R3 receptor with a test compound in both the absence and in the presence of pyrophosphate, wherein the compound has a pyrophosphate-enhanced activity on the receptor if the response of the receptor to the compounds in a mixture in which each compound is presented at a given concentration is greater than the response to one of the compounds alone at that given concentration. In further preferred embodiments, the cells are transfected with one or more polynucleotides encoding the T1R1/T1R3 receptor.

Provided is a method for determining the pyrophosphate-suppressive activity of a test compound on the T1R1/T1R3 receptor comprising contacting one or more cells expressing the T1R1/T1R3 receptor with a test compound in both the absence and in the presence of pyrophosphate, wherein the compound has a suppressive activity if the response to the compound and pyrophosphate is less than the smaller response to either the test compound or the pyrophosphate alone.

In preferred embodiments of the previous two methods, the T1R1/T1R3 receptor is a Felidae T1R1/T1R3 receptor. In yet further preferred embodiments, the response of the T1R1/T1R3 receptor was measured by calcium imaging. In preferred embodiments, the cells are HEK293-Gα15 cells. In further preferred embodiments, the pyrophosphate is present at a final concentration equal to or greater than 1 mM. In further preferred embodiments, the pyrophosphate is present at a final concentration equal to or greater than 5 mM. In further preferred embodiments, the pyrophosphate is present at a final concentration equal to or greater than 10 mM. In further preferred embodiments, the pyrophosphate is present at a final concentration equal to or greater than 20 mM. In yet further preferred embodiments, the pyrophosphate is present at a final concentration equal to or greater than 25 mM. In preferred embodiments, the pyrophosphate is sodium pyrophosphate. In further preferred embodiments, the pyrophosphate is trisodium pyrophosphate, or Na₃HP₂O₇.

Provided is a method for adjusting a food formulation for Felidae comprising contacting one or more cells transfected with T1R1/T1R3 receptor with a test compound in both the absence and in the presence of pyrophosphate, conducting calcium imaging to measure the response of the T1R1/T1R3 receptor in the cells, wherein the compounds have an augmenting effect on the receptor if the response of the receptor to the compounds in a mixture in which each compound is presented at a given concentration is greater than the response to one of the compounds alone at its initial concentration, if each test compound alone elicits a small or moderate response, adding both the test compound that has been determined to exhibit pyrophosphate augmented activity and pyrophosphate to a current food formulation to create an adjusted food formulation, measuring the preference of Felidae for the adjusted food formulation containing the test compound and pyrophosphate, compared to the preference of the Felidae for the current food formulation without the added test compound and pyrophosphate, wherein if the Felidae show a preference for the adjusted food formulation containing the test compound and pyrophosphate, compared to the current food formulation without the added test compound and pyrophosphate, the test compound and pyrophosphate is selected for addition to food formulations for the Felidae. In preferred embodiments, the cells are HEK293-Gα15 cells. In preferred embodiments, the pyrophosphate is present at a final concentration equal to or greater than 1 mM. In further preferred embodiments, the pyrophosphate is present at a final concentration equal to or greater than 5 mM. In further preferred embodiments, the pyrophosphate is present at a final concentration equal to or greater than 10 mM. In yet further preferred embodiments, the pyrophosphate is present at a final concentration equal to or greater than 20 mM. In further preferred embodiments, the pyrophosphate is present at a final concentration equal to or greater than 25 mM.

Provided is a method for determining the minimal amount of a test compound and pyrophosphate that are needed to augment the taste intensity of a food formulation for Felidae comprising contacting the T1R1/T1R3 receptor with a test compound in both the absence and in the presence of pyrophosphate, measuring the response of the T1R1/T1R3 receptor to the test compound in both the absence and in the presence of pyrophosphate, wherein if an increase in the response of the T1R1/T1R3 receptor is measured in the presence of both the test compound and pyrophosphate relative to the response of the T1R1/T1R3 receptor measured in the presence of test compound or pyrophosphate alone, the test compound is determined to exhibit pyrophosphate augmented activity, adding both the test compound that has been determined to exhibit pyrophosphate augmented activity and pyrophosphate to a current food formulation to create a taste intensity augmented food formulation, measuring the preference of Felidae for the taste intensity augmented food formulation containing the test compound and pyrophosphate, compared to the preference of the Felidae for the current food formulation without the added test compound and pyrophosphate, wherein if the Felidae show a preference for the taste intensity augmented food formulation containing the test compound and pyrophosphate, compared to the current food formulation without the added test compound and pyrophosphate, the test compound and pyrophosphate is selected for addition to food formulations for the Felidae. The method is repeated with consecutively decreasing amounts of test compound and pyrophosphate, until the minimum quantity of test compound and pyrophosphate needed for the Felidae to demonstrate a preference for the adjusted food formulation containing the test compound and pyrophosphate, compared to the current food formulation without the added test compound and pyrophosphate is determined.

Also provided is a method for augmenting the animal equivalent of human umami taste in domestic animal foodstuff by adding pyrophosphate to domestic animal foodstuff comprising one or more amino acids. In preferred embodiments, the one or more amino acids are Lysine, Alanine, Proline or Histidine. In further preferred embodiments, the domestic animal foodstuff is a catfood.

Provided herein are methods for stimulating the T1R1/T1R3 receptor comprising contacting the receptor with pyrophosphate and one or more amino acids. In some embodiments, the receptor is first contacted with pyrophosphate. In some embodiments, the receptor is first contacted with one or more amino acids. In preferred embodiments, an excess of receptor is contacted with pyrophosphate. In some embodiments, the pyrophosphate may be present at a final concentration equal to or greater than 1 mM. In some embodiments, the pyrophosphate may be present at a final concentration equal to or greater than 5 mM. In further embodiments, the pyrophosphate may be present at a final concentration equal to or greater than 10 mM. In further preferred embodiments, the receptor is contacted with pyrophosphate present at a final concentration equal to or greater than 20 mM. In yet further preferred embodiments, the receptor is contacted with pyrophosphate present at a final concentration equal to or greater than 25 mM. In some preferred embodiments, the one or more amino acids is Lysine, Alanine, Proline or Histidine. In some preferred embodiments, the T1R1/T1R3 receptor is a Felidae T1R1/T1R3 receptor. In further preferred embodiments, the T1R1/T1R3 receptor comprises the cat T1R1 and the cat T1R3. The sequences of the corresponding polypeptides and cDNAs may be found in the attached sequence listing and are listed below:

cat T1R1 polypeptide amino acid sequence: SEQ ID NO:1

cat T1R3 polypeptide amino acid sequence: SEQ ID NO:2

cat T1R1 cDNA sequence: SEQ ID NO:3

cat T1R3 cDNA sequence: SEQ ID NO:4

Provided herein is a Felidae food product comprising pyrophosphate and one or more amino acids. In preferred embodiments the food product contains pyrophosphate present at a final concentration that provides a final salivary concentration equal to or greater than 1 mM. In some preferred embodiments the food product contains pyrophosphate present at a final concentration that provides a final salivary concentration equal to or greater than 5 mM. In further preferred embodiments the food product contains pyrophosphate present at a final concentration that provides a final salivary concentration equal to or greater than 10 mM. In yet further preferred embodiments, the food product contains pyrophosphate present at a final concentration that provides a final salivary concentration equal to or greater than 20 mM. In yet further preferred embodiments, the food product contains pyrophosphate present at a final concentration that provides a final salivary concentration equal to or greater than 25 mM. In preferred embodiments, the pyrophosphate is sodium pyrophosphate. In preferred embodiments, the one or more amino acids are Lysine, Alanine, Proline or Histidine. In further embodiments, non-naturally occurring analogs or mimetics of said amino acids may be employed.

Provided herein is a method for augmenting taste intensity of a cat food by adding pyrophosphate and one or more amino acids in a determined pyrophosphate: amino acid ratio. In preferred embodiments, the pyrophosphate: amino acid ratio is determined by the method described supra for determining the minimal amount of a test compound and pyrophosphate that are needed to augment the taste intensity of a food formulation for Felidae.

Also provided is a cat food product obtainable by the method described supra for augmenting taste intensity of a cat food by adding pyrophosphate and one or more amino acids in a determined pyrophosphate: amino acid ratio.

Provided is a taste intensity augmenting agent for cat food comprising pyrophosphate and one or more amino acids in a determined pyrophosphate: amino acid ratio. In preferred embodiments, the pyrophosphate: amino acid ratio is determined by the method described supra for determining the minimal amount of a test compound and pyrophosphate that are needed to augment the taste intensity of a food formulation for Felidae.

### BRIEF DESCRIPTION OF THE DRAWINGS

FIG. 1 illustrates that an HEK293 cell expressing T1R1/T1R3 receptor and Gα15 gave null responses to 25 mM L-proline (L-Pro) alone as well as mixtures of 25 mM L-proline plus 1, 5, or 10 mM sodium pyrophosphate (PP) but exhibited a robust response to a mixture of the two compounds (25 mM L-proline plus 20 mM sodium pyrophosphate). The augmented response was replicable. 100 μM isoproterenol serves as positive control. The pyrophosphate employed was trisodium pyrophosphate, or Na₃HP₂O₇.

FIG. 2 illustrates that 3 HEK293 cells expressing T1R1/T1R3 receptor and Gα15 gave null responses to 50 mM L-proline (L-Pro) alone as well as mixtures of 50 mM L-proline plus 1, 5, or 10 mM sodium pyrophosphate (PP) but exhibited a robust response to a mixture of the two compounds (50 mM L-proline plus 20 mM pyrophosphate). 100 μM isoproterenol serves as positive control.

FIG. 3 illustrates that an HEK293 cell expressing T1R1/T1R3 receptor and Gα15 gave null responses to 50 mM L-proline (L-Pro), 10 mM and 20 mM sodium pyrophosphate (PP) presented singly but exhibited a robust response to a mixture of the two compounds (50 mM L-proline plus 20 mM sodium pyrophosphate). 100 μM isoproterenol serves as positive control.

FIG. 4 illustrates that 5 HEK293 cells expressing T1R1/T1R3 receptor and Gα15 gave null responses to 25 mM L-histidine (L-His), 20 mM sodium pyrophosphate(PP) and 25 mM L-lysine (L-Lys) alone but exhibited a robust response to a mixture of 25 mM L-lysine plus 20 mM sodium pyrophosphate. L-histidine was not augmented in this study. 100 μM isoproterenol serves as positive control.

FIG. 5 illustrates that a HEK293 cell expressing T1R1/T1R3 receptor and Gα15 gave null responses to 25 mM L-histidine (L-His) and L-alanine (L-Ala) alone as well as mixtures of 25 mM L-histidine (L-His) plus 20 mM sodium pyrophosphate (PP) but exhibited a robust response to a mixture of 25 mM L-alanine (L-Ala) plus 20 mM sodium pyrophosphate (PP). 100 μM isoproterenol serves as positive control.

FIG. 6 illustrates that a HEK293 cell expressing T1R1/T1R3 receptor and Gα15 gave a weak response to 20 mM sodium pyrophosphate (PP) alone and a null response to 25 mM L-lysine (L-Lys) alone but exhibited a robust response to a mixture of 20 mM sodium pyrophosphate (PP) and 25 mM L-lysine (L-Lys). 100 μM isoproterenol serves as positive control.

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

All references cited herein are incorporated by reference in their entirety and for all purposes.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “pyrophosphate” as used herein refers to a compound with a P₂O₇ anion. In preferred embodiments, the pyrophosphate is sodium pyrophosphate. Pyrophosphates are available as di-, tri- and tetra-sodium salts (Na₂H₂P₂O₇, Na₃HP₂O₇, and Na₄P₂O₇, respectively), as well as several non-sodium forms. In the disclosed examples, the pyrophosphate employed was trisodium pyrophosphate, or Na₃HP₂O₇.

The term “Felidae” as used herein refers to a cosmopolitan family comprising lithe-bodied carnivorous mammals having often strikingly patterned fur, comparatively short limbs with soft pads on the feet, usually sharp curved retractile claws, a broad and somewhat rounded head with short but powerful jaws equipped with teeth suited to grasping, tearing, and shearing through flesh, erect ears, and typically eyes with narrow or elliptical pupils and especially adapted for seeing in dim light. Examples of Felidae comprise the domestic cat, lion, tiger, lynx, leopard, wildcat, saber-toothed tiger and cheetah.

The term “Felidae T1R1/T1R3 receptor” as used herein refers to a heterodimer of Felidae T1R1 and T1R3 polypeptides. One example of a Felidae T1R1 polypeptide comprises the amino acid sequence SEQ ID NO: 1. An example of a Felidae T1R3 polypeptide comprises the amino acid sequence SEQ ID NO: 2. The exemplary Felidae T1R1 polypeptide may be encoded by the nucleotide sequence of SEQ ID NO: 3. The exemplary Felidae T1R3 polypeptide may be encoded by the nucleotide sequence of SEQ ID NO: 4. The term “Felidae T1R1 polypeptide” refers to polymorphic variants, alleles, mutants, and interspecies homologs that: (1) have at least about 90, 95, 96, 97, 98, or 99% amino acid sequence identity to SEQ ID NO: 1; (2) specifically bind to antibodies raised against an immunogen comprising an amino acid sequence of SEQ ID NO: 1 and conservatively modified variants thereof; or (3) are encoded by a nucleic acid molecule which specifically hybridize (with a size of at least about 100, optionally at least about 500-1000 nucleotides) under stringent hybridization conditions to a sequence of SEQ ID NO: 3 and conservatively modified variants thereof The term “Felidae T1R3 polypeptide” refers to polymorphic variants, alleles, mutants, and interspecies homologs that: (1) have at least about 90, 95, 96, 97, 98, or 99% amino acid sequence identity to SEQ ID NO: 2; (2) specifically bind to antibodies raised against an immunogen comprising an amino acid sequence of SEQ ID NO: 2 and conservatively modified variants thereof; or (3) are encoded by a nucleic acid molecule which specifically hybridize (with a size of at least about 100, optionally at least about 500-1000 nucleotides) under stringent hybridization conditions to a sequence of SEQ ID NO: 4 and conservatively modified variants thereof.

The term “amino acid” as used herein denotes a molecule containing both an amino group and a carboxyl group. In some embodiments, the amino acids are α-, β-, γ- or δ-amino acids, including their stereoisomers and racemates. As used herein the term “L-amino acid” denotes an a-amino acid having the L configuration around the α-carbon, that is, a carboxylic acid of general formula CH(COOH)(NH₂)-(side chain), having the L-configuration. The term “D-amino acid” similarly denotes a carboxylic acid of general formula CH(COOH)(NH₂)-(side chain), having the D-configuration around the a-carbon. Side chains of L-amino acids include naturally occurring and non-naturally occurring moieties. Non-naturally occurring (i.e., unnatural) amino acid side chains are moieties that are used in place of naturally occurring amino acid side chains in, for example, amino acid analogs. Amino acid substituents may be attached, for example, through their carbonyl groups through the oxygen or carbonyl carbon thereof, or through their amino groups, or through functionalities residing on their side chain portions. The naturally occurring amino acids are listed in Table 1 and the unnaturally occurring amino acids are listed in Table 2 below.

TABLE 1
Naturally occurring amino acids.

| Symbol | Meaning |
|---|---|
| Ala | Alanine |
| Cys | Cysteine |
| Asp | Aspartic Acid |
| Glu | Glutamic Acid |
| Phe | Phenylalanine |
| Gly | Glycine |
| His | Histidine |
| IIe | Isoleucine |
| Lys | Lysine |
| Leu | Leucine |
| Met | Methionine |
| Asn | Asparagine |
| Pro | Proline |
| Gln | Glutamine |
| Arg | Arginine |
| Ser | Serine |
| Thr | Threonine |
| Val | Valine |
| Trp | Tryptophan |
| Tyr | Tyrosine |

TABLE 2
Unnaturally occurring amino acids

| Symbol | Meaning |
|---|---|
| Aad | 2-Aminoadipic acid |
| bAad | 3-Aminoadipic acid |
| bAla | beta-Alanine, beta-Aminopropionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| bAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylgycine |
| EtAsn | N-Ethylasparagine |
| Hyl | Hydroxylysine |
| aHyl | Allo-hydroxylysine |
| 3Hyp | 3-Hydroxyproline |
| 4Hyp | 4-Hydroxyproline |
| Ide | Isodesmosine |
| Alle | Allo-Isoleucine |
| MeGly | N-Methylglycine, sarcosine |
| Melle | N-Methylisoleucine |
| MeLys | 6-N-Methyllysine |
| MeVal | N-Methylvaline |
| Nva | Norvaline |
| Nle | Norleucine |
| Orn | Ornithine |

The term “null response” as used herein refers to a response that is less than 10% of the response elicited by the positive control. In preferred embodiments, the positive control is isoproterenol.

The term “small or moderate response” as used herein refers to a response that is equal to or greater than 10% of the responses elicited by the positive control. In preferred embodiments, the positive control is isoproterenol.

The term “augmenting effect” of a compound as used herein refers to the effect of a compound which when mixed at its initial concentration with another compound at its initial concentration gives a greater response than the response to either one of the compounds alone at their initial concentrations.

The term “domestic animal foodstuff' as used herein refers to commercially available solid wet or dry food for consumption by domestic companion animals

It is to be understood that the embodiments described herein are not limited to particular methods, reagents, compounds, compositions or biological systems, which can, of course, vary.

The cells employed in certain embodiments of the invention are cell lines that were transfected with or express one or more polynucleotides encoding the T1R1/T1R3 receptor and that also were transfected with or express a G protein such as Gα15 or another G protein that when expressed in association with the T1R1/T1R3 receptor produces a functional taste receptor. Cell lines that may be employed include HEK293, CHO, Jurkat, HeLa, Sf9 or other expression cell lines. The cells may be stably or transiently transfected, although stable transfection is preferred. Preferably, the cells are mammalian cells and the T1R1/T1R3 receptor is Felidae. In preferred embodiments the cells are HEK293-Gα15 cells transfected with Felidae T1R1/T1R3 receptor. The T1R1/T1R3 cDNA and protein sequences from various species, including Felidae, are provided in the sequence listing.

The responses of cells to taste stimuli are measured by using calcium imaging techniques. Calcium imaging techniques are scientific techniques that show the calcium status of a tissue, cell or medium (Nelson et al. 2001, Li et al. 2002 and Gomez et al. 2005). Transfected cells are loaded with a calcium indicator containing buffer. Preferably, the calcium indicator is a fluorescent dye, including but not limited to acetoxymethyl esters, for example Calcium Green 1-AM™, Fura2-AM™, and Fura Red AM™. In preferred embodiments, the calcium indicator is Fura2-AM™. Stimulus solutions containing the test compounds, such as amino acids and/or pyrophosphate, are applied to the cells, and calcium imaging is conducted. Preferably, coverslips with Fura2-AM™ loaded cells are placed in a recording chamber and continuously superfused with Hank's salt solution (Nelson et al. 2001) and stimulus solutions are applied by switching the superfusion. Other techniques and instruments that may be appropriate measure the calcium status of either many individual receptor-expressing cells or the calcium status of a population of receptor-expressing cells.

The methods of the invention may be used to identify test compounds that have an augmenting effect on the T1R1/T1R3 receptor, as described in the Summary of the invention. Once a test compound or a combination of test compounds has been identified that have an augmenting effect on the T1R1/T1R3 receptor, those test compounds may be selected for addition to current food formulations for Felidae, to create an adjusted food formulation. The preference of Felidae for the adjusted food formulation is measured by behavioral methods. Exemplary behavioral methods for determining preference for an adjusted food formulation include a two-bottle preference test, a lickometer, or a conditioned aversion assay. The consumption by Felidae of the adjusted food formulation is compared to the consumption by Felidae of the current food formulation without the added test compounds. If the Felidae show a preference for the adjusted food formulation containing the test compounds, compared to the current food formulation without the added test compounds, the test compounds are selected for addition to food formulations for the Felidae. In preferred embodiments, the test compounds comprise one or more amino acids and pyrophosphate.

Once a test compound has been determined to exhibit pyrophosphate augmented activity by the methods described in the summary of the invention, the method may be repeated with consecutively decreasing amounts of test compound and pyrophosphate, until the minimum quantity of test compound and pyrophosphate needed for the Felidae to demonstrate a preference for the adjusted food formulation over the food formulation without the added test compound and pyrophosphate is determined.

### EXAMPLES

### Example 1

Constructing the Heterologous Cell Lines

Sequencing of Cat Tas1r1 and Tas1r3.

The coding sequences of cat Tas1r1 and cat Tas1r3 were identified by screening Felidae Genomic BAC library. The procedures for library screening and BAC sequencing were described in (Li et al. 2002; Li et al. 2005).

Initiating Heterologous Expression.

a. cDNA constructs: To generate the full-length T1R expression constructs from cat, a PCR overlapping strategy was used to amplify coding regions of the Tas1r3 gene (Horton et al. 1990). The full-length cDNA clone of Tas1r1 was obtained by screening a cat taste cDNA library. The methods for designing overgo probes and procedures for cDNA library screening were previously described (Li et al. 2005). The gene for T1R1 was cloned into expression vector pcDNA4/HisMax, and the gene for the T1R3 receptor was cloned into pCEP4. These two expression vectors have different antibiotic selections so that cells expressing receptor dimer, e.g., T1R1/T1R3, can be selected and characterized.

b. Cell Transfection: HEK293 cell lines that stably express Gα15 were grown and maintained at 37° C. DMEM supplemented with 10% FBS and MEM nonessential amino acids (Invitrogen); the medium for Gα15 transfected cells contains 5 μg/ml blasticidin (Invitrogen). Cells were co-transfected with expression constructs by using Lipofectamine 2000 reagent (Invitrogen). T1R1/T1R3 transfected cells were selected using Zeocin (100 μg/ml) and hygromycin (400 μg/ml).

c. Establishing stable cell lines: Transfected cells were maintained in antibiotic selective medium until colonies appear. Single colonies were identified two weeks after transfection and were examined for RNA expression by RT-PCR and for protein expression by immunohistochemistry. Positive colonies confirmed by RT-PCR and immunohistochemistry were stocked in liquid nitrogen as stable cell lines expressing T1R1/T1R3.

### Example 2

Method for Detecting Pyrophosphate Enhancement.

a. In vitro assays: HEK293 cells' responses to taste stimuli were measured using calcium imaging techniques (Nelson et al. 2001, Li et al. 2002, Gomez et al. 2005). Transfected cells were loaded with fura-2 containing buffer for one hour. Coverslips with fura-2 loaded cells were then placed in a recording chamber and continuously superfused with Hank's salt solution (Nelson et al. 2001). Enhancement solutions were applied by switching the superfusion, which allowed a complete change of bath solutions within 5-20 seconds, based on preliminary experiments with fluorescent dye for each setup and recording chamber. Calcium imaging recordings were performed using Imagemaster-5 multi-wavelength quantitative fluorescence imaging system (Photon Technology International, Inc.). Cells were illuminated with UV light at two wavelengths (340 or 380 nm). Intracellular calcium activity was computed from the ratio of emitted light from the 340/380 nm wavelengths. Excitation light was shuttered between acquisitions, with exposure times being minimized (typically 50-100 msec). Images are normally acquired every 2-5 sec. Using these conditions, cells remain viable in the recording setup for more than 2 h without significant effects of dye bleaching or baseline shifts. The use of ratiometric imaging reduces the problems associated with fluorescence quenching or dye loss that can generate artifacts using single-wavelength dyes.

b. Amino acid stimulation of the HEK transfected cell lines: In a typical experiment to determine if there is an interaction at the receptor between two compounds, the response (peak height of intracellular calcium concentration) to each compound as well as a mixture of the two compounds is measured. If each compound alone elicits a small or moderate response, it will be necessary to compare responses to single compounds with a mixture in which each compound is presented at its initial test concentration. In this manner we can determine if the enhancement is greater than simple additivity. Suppressive interactions are simpler to detect; the response to a mixture will be less than the smaller response to each single compound. If either suppression or enhancement is detected, control experiments are carried out to rule out self-adaptation or self-sensitization.

### Example 3

Results

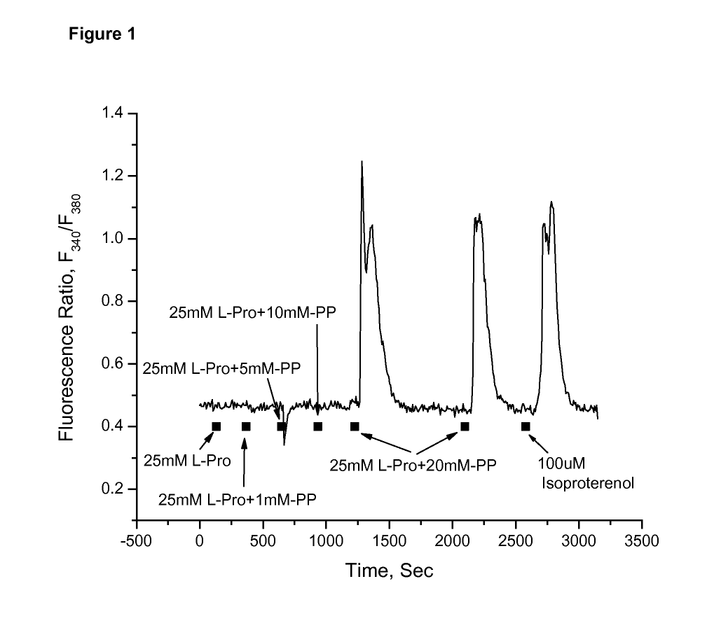

The results of the HEK293 cells' responses to taste stimuli are shown in the Figures. FIG. 1 illustrates that an HEK cell expressing T1R1/T1R3 receptor and Gα15 gave null responses to 25 mM L-proline (L-Pro) alone as well as mixtures of 25 mM L-proline plus 1, 5, or 10 mM sodium pyrophosphate (PP) but exhibited a robust response to a mixture of the two compounds (25 mM L-proline plus 20 mM sodium pyrophosphate). The augmented response was replicable. 100 μM isoproterenol serves as positive control. The pyrophosphate employed was trisodium pyrophosphate, or Na₃HP₂O₇.

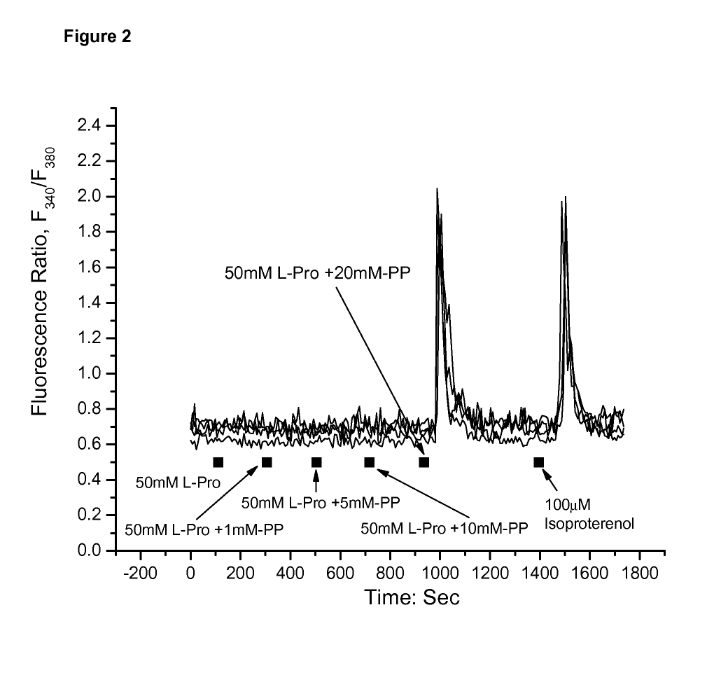

FIG. 2 illustrates that 3 HEK293 cells expressing T1R1/T1R3 receptor and Gα15 gave null responses to 50 mM L-proline (L-Pro) alone as well as mixtures of 50 mM L-proline plus 1, 5, or 10 mM sodium pyrophosphate (PP) but exhibited a robust response to a mixture of the two compounds (50 mM L-proline plus 20 mM pyrophosphate). 100 μM isoproterenol serves as positive control.

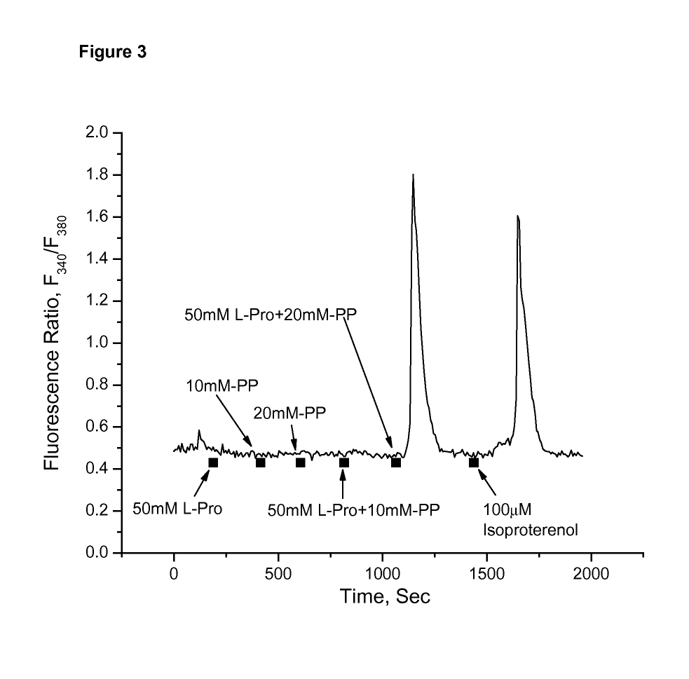

FIG. 3 illustrates that an HEK293 cell expressing T1R1/T1R3 receptor and Gα15 gave null responses to 50 mM L-proline (L-Pro), 10 mM and 20 mM sodium pyrophosphate (PP) presented singly but exhibited a robust response to a mixture of the two compounds (50 mM L-proline plus 20 mM sodium pyrophosphate). 100 μM isoproterenol serves as positive control.

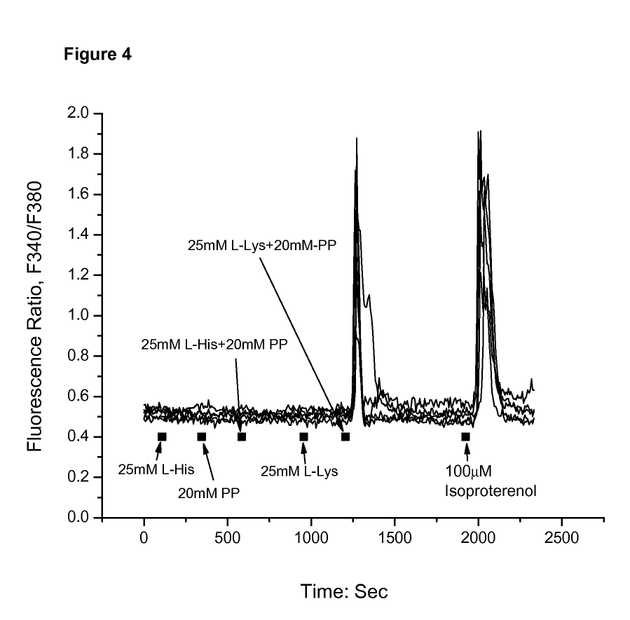

FIG. 4 illustrates that 5 HEK cells expressing T1R1/T1R3 receptor and Gα15 gave null responses to 25 mM L-histidine (L-His), 20 mM sodium pyrophosphate(PP) and 25 mM L-lysine (L-Lys) alone but exhibited a robust response to a mixture of 25 mM L-lysine plus 20 mM sodium pyrophosphate. L-histidine was not augmented in this study. 100 μM isoproterenol serves as positive control.

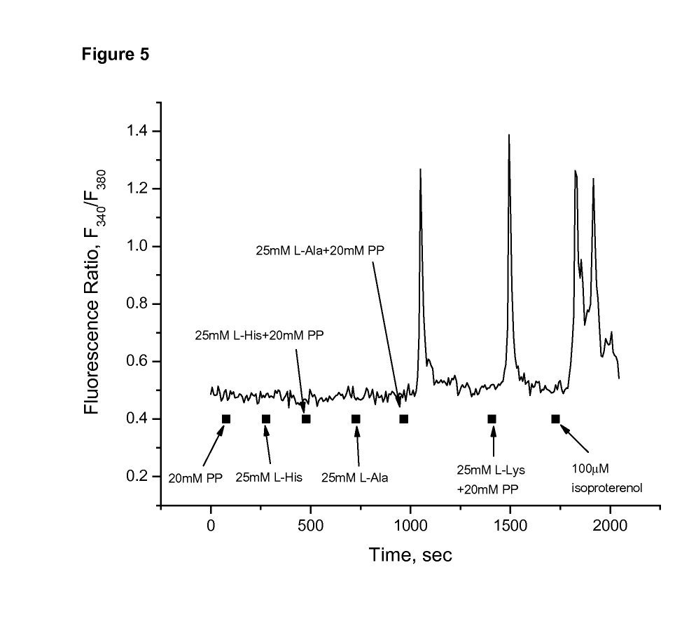

FIG. 5 illustrates that a HEK293 cell expressing T1R1/T1R3 receptor and Gα15 gave null responses to 25 mM L-histidine (L-His) and L-alanine (L-Ala) alone as well as mixtures of 25 mM L-histidine (L-His) plus 20 mM sodium pyrophosphate (PP) but exhibited a robust response to a mixture of 25 mM L-alanine (L-Ala) plus 20 mM sodium pyrophosphate (PP). 100 μM isoproterenol serves as positive control.

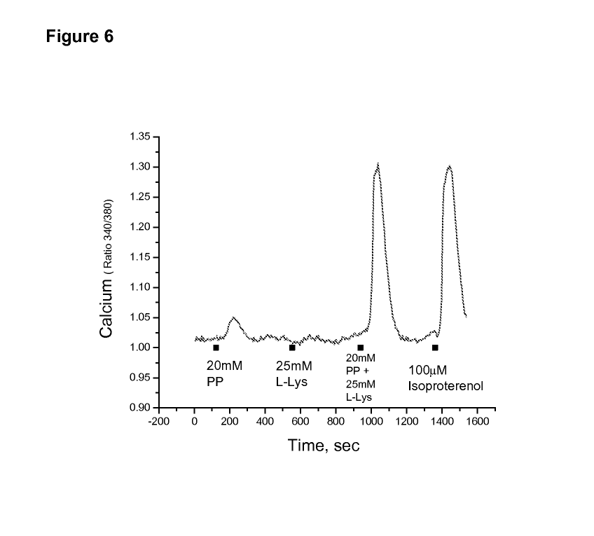

FIG. 6 illustrates that a HEK293 cell expressing T1R1/T1R3 receptor and Gα15 gave a weak response to 20 mM sodium pyrophosphate (PP) alone and a null response to 25 mM L-lysine (L-Lys) alone but exhibited a robust response to a mixture of 20 mM sodium pyrophosphate (PP) and 25 mM L-lysine (L-Lys).

### REFERENCES

Gomez G, Lischka F W, Haskins M E, Rawson N E. 2005. Evidence for multiple calcium response mechanisms in mammalian olfactory receptor neurons. Chem Senses 30: 317-26.

Horton R M, Cai Z L, Ho S N, Pease L R. 1990. Gene splicing by overlap extension: tailor-made genes using the polymerase chain reaction. Biotechniques 8: 528-35.

Li X, Bachmanov A A, Li S, Chen Z, Tordoff M G, Beauchamp G K, de Jong P J, Wu C, Chen L, West D B, Ross D A, Ohmen J D, Reed D R. 2002. Genetic, physical, and comparative map of the subtelomeric region of mouse Chromosome 4. Mamm Genome 13: 5-19.

Li X, Li W, Wang H, Cao J, Maehashi K, Huang L, Bachmanov A A, Reed D R, Legrand-Defretin V, Beauchamp G K, Brand J G. 2005. Pseudogenization of a sweet-receptor gene accounts for cats' indifference toward sugar. PLoS Genet 1: 27-35.

McCaughey, S. A., Giza, B. K., & Tordoff, M. G. (2007). Taste and acceptance of pyrophosphates by rats and mice. American Journal of Physiology, 292, R2159-R2167

Nelson G, Hoon M A, Chandrashekar J, Zhang Y, Ryba N J, Zuker C S. 2001. Mammalian sweet taste receptors. Cell 106: 381-90.

1-48. (canceled)

49. A method for determining if two or more test compounds have an augmenting effect on the T1R1/T1R3 receptor comprising:
contacting said receptor with said test compounds;
measuring the response of said receptor to said test compounds;
wherein said compounds have an augmenting effect on said receptor if the response of said receptor to said compounds in a mixture in which each compound is presented at a given concentration is greater than the response to one of the compounds alone at its given respective concentration, if each test compound alone elicits a small or moderate response.

50. The method of claim 49 wherein said T1R1/T1R3 receptor is a Felidae T1R1/T1R3 receptor.

51. The method of claim 49 wherein one or more said test compounds is an aminoacid.

52. The method of anyone of claim 49 wherein one said test compound is pyrophosphate.

53. A method for selecting a cell line that is suitable for determining if two or more test compounds have an augmenting effect on the T1R1/T1R3 receptor comprising:
contacting a cell line with isoproterenol;
conducting calcium imaging to measure the response of the endogenous isoproterenol receptor in said cell line;
wherein if a response of the endogenous isoproterenol receptor is observed, said cell line is selected for further analysis.

54. A method for generating a cell line expressing the T1R1/T1R3 receptor comprising:
contacting a cell line with isoproterenol;
conducting calcium imaging to measure the response of the endogenous isoproterenol receptor in said cell line;
selecting a cell line that responds to isoproterenol; and
transfecting the selected cell line with one or more polynucleotides encoding the T1R1/T1R3 receptor.

55. A method for determining the pyrophosphate-augmented activity of a test compound on the T1R1/T1R3 receptor comprising:
contacting a cell expressing the T1R1/T1R3 receptor with a test compound in both the absence and in the presence of pyrophosphate;
wherein said compounds have an augmenting effect on said receptor if the response of said receptor to said compounds in a mixture in which each compound is presented at 50% of its initial concentration is greater than the response to one of the compounds alone at its initial concentration, if each test compound alone elicits a small or moderate response.

56. A method for determining the pyrophosphate-suppressive activity of a test compound on the T1R1/T1R3 receptor comprising:
contacting a cell expressing the T1R1/T1 R3 receptor with a test compound in both the absence and in the presence of pyrophosphate;
wherein said compounds have a suppressive activity if the response to the mixture is less than the smaller response to each single compound.

57. A method for adjusting a food formulation for Felidae comprising:
contacting a cell transfected with T1R1/T1R3 receptor with a test compound in both the absence and in the presence of pyrophosphate;
conducting calcium imaging to measure the response of the T1R1/T1R3 receptor in said cells;
wherein said compounds have an augmenting effect on said receptor if the response of said receptor to said compounds in a mixture in which each compound is presented at 50% of its initial concentration is greater than the response to one of the compounds alone at its initial concentration, if each test compound alone elicits a small or moderate response;
adding both said test compound that has been determined to exhibit pyrophosphate augmented activity and pyrophosphate to a current food formulation to create an adjusted food formulation;
measuring the preference of Felidae for said adjusted food formulation containing the test compound and pyrophosphate, compared to the preference of said Felidae for the current food formulation without the added test compound and pyrophosphate;
wherein if said Felidae show a preference for said adjusted food formulation containing the test compound and pyrophosphate, compared to the current food formulation without the added test compound and pyrophosphate, said test compound and pyrophosphate is selected for addition to food formulations for said Felidae.

58. A method for determining the minimal amount of a test compound and pyrophosphate that are needed to augment the taste intensity of a food formulation for Felidae comprising:
contacting the T1R1/T1R3 receptor with a test compound in both the absence and in the presence of pyrophosphate;
measuring the response of the T1R1/T1R3 receptor to said test compound in both the absence and in the presence of pyrophosphate;
wherein if an increase in the response of the T1 R1/T1 R3 receptor is measured in the presence of both the test compound and pyrophosphate relative to the response of the T1R1/T1R3 receptor measured in the presence of test compound or pyrophosphate alone, said test compound is determined to exhibit pyrophosphate augmented activity;
adding both said test compound that has been determined to exhibit pyrophosphate augmented activity and pyrophosphate to a current food formulation to create a taste intensity augmented formulation;
measuring the preference of Felidae for said taste intensity augmented food formulation containing the test compound and pyrophosphate, compared to the preference of said Felidae for the current food formulation without the added test compound and pyrophosphate;
wherein if said Felidae show a preference for said adjusted food formulation containing the test compound and pyrophosphate, compared to the current food formulation without the added test compound and pyrophosphate, said test compound and pyrophosphate is selected for addition to food formulations for said Felidae; and
wherein said method is repeated with consecutively decreasing amounts of test compound and pyrophosphate, until the minimum quantity of test compound and pyrophosphate needed for the Felidae to demonstrate a preference for said adjusted food formulation containing the test compound and pyrophosphate, compared to the current food formulation without the added test compound and pyrophosphate is determined.

59. A method for augmenting the domestic animal equivalent to human umami taste in domestic animal foodstuff by adding pyrophosphate to domestic animal foodstuff.

60. A method for stimulating the T1R1/T1R3 receptor comprising:
contacting said receptor with pyrophosphate and one or more amino acids.

61. A Felidae food product comprising:
pyrophosphate and one or more amino acids.

62. A method for augmenting taste intensity of a cat food by adding pyrophosphate and one or more amino acids in a determined pyrophosphate: amino acid ratio.

63. A cat food product obtainable by the method of claim 62.

64. A taste intensity augmenting agent for cat food comprising pyrophosphate and one or more amino acids in a determined pyrophosphate: amino acid ratio.


SEQUENCE LISTING (4 sequences)

SEQ ID NO: 1 - protein, 841 aa, Felis catus
MSLPAAHLVGLQLSLSCCWALSCHSTETSADFSLPGDYLLAGLFPLHSDCPGVRHRPTVTLCDRPDSFNGHGYHLFQAMRFGIEEINNSTALLPNVTLGYQLYDVCSESANVYATLNVLSLLGTHHVEIRADPSHYSPAALAVIGPDTTNHAATTAALLSPFLVPLISYEASSVTLGVKRHYPSFLRTIPSDKHQVEAMVLLLQSFGWVWISVVGSDGDYGQLGVQALEEQATQQGICVAFKDIIPFSARPGDERMQSIMHHLARARTTVVVVFSSRQLARVFFESVVLANLTAKVWIASEDWAISRHISNVPGIQGIGTVLGVAIQQRLVPGLKEFEEAYVQADKGAPGPCSRTSECSSNQLCRECRAFTAEQMPTLGAFSMSSAYNAYRAVYAVAHGLHQLLGCASGACSRDRVYPWQLLEQIRKVNFLLHKDTVRFNDNGDPLSGYDIIAWDWSGPKWNFRVIGSSMWPPVQLDINKTKIRWHGKDNQVPKSVCSSDCLEGHQRVISGFYHCCFECVPCEAGSFLNKSDLHSCQPCGKEKWAPAGSETCFPRTVVFLTWHETISWVLLAANTLLLLLVTGTAGLFAWHLDTPVVKSAGGRLCFFMLGSLAGGSCGLYGFFGEPTLPTCLLRQSLLALGFAIFLSCLTIRSFQLVFIFKFSAKVPTFYRAWVQNHGPGLFVVISSMAQLLICLTWLAVWTPLPTREYQRFPQLVVLDCTEANSPGFMLAFAYNGLLSVSAFACSYLGKDLPENYNEAKCVTFSLLLNFVSWIAFFTTASVYQGKYLPAVNVLAALSSLSGGFSGYFLPKCYVILCRPKFNSTQHFQASIQEYTRRCGST

SEQ ID NO: 2 - protein, 865 aa, Felis catus
MPGLALLGLTALLGLTALLDHGEGATSCLSQQLRMQGDYVLGGLFPLGSAEGTGLGDGLQPNATVCTRFSSLGLLWALAVKMAVEEINNGSALLPGLHLGYDLFDTCSEPMVAMKPSLVFMAKAGSCSIAAYCNYTQYQPRVLAVIGPHSSELALVTGKFFSFFLVPQVSYGASTDRLSNREIFPSFFRTVPSDQVQVAAMVELLEELGWNWVAAVGSDDEYGRQGLSLFSGLASARGICIAHEGLVPLPPGSLRLGALQGLLRQVNQSSVQVVVLFSSAHAARTLFSYSIRCKLSPKVWVASEAWLTSDLVMTLPGMPGVGTVLGFLQQGAPMPEFPSYVRTRLALAADPAFCASLDAEQPGLEEHVVGPRCPQCDHVTLENLSAGLLHHQTFAAYAAVYGVAQALHNTLRCNASGCPRREPVRPWQLLENMYNVSFRARGLALQFDASGNVNVDYDLKLWVWQDPTPELRTVGTFKGRLELWRSQMCWHTPGKQQPVSQCSRQCKEGQVRRVKGFHSCCYNCVDCKAGSYQRNPDDLLCTQCDQDQWSPDRSTRCFARKPMFLAWGEPAVLLLLALLALALGLALAALGLFLWHSDSPLVQASGGPRACFGLACLGLVCLSVLLFPGQPGPASCLAQQPLFHLPLTGCLSTFFLQAAEIFVGSELPPSWAEKMRGRLRGPWAWLVVLLAMLAEAALCAWYLVAFPPEVVTDWRVLPTEALVHCHVHSWISFGLVHATNAMLAFLCFLGTFLVQSRPGRYNGARGLTFAMLAYFITWISFVPLFANVHVAYQPAVQMGTILLCALGILATFHLPKCYLLLQRPELNTPEFFLEDNARAQGSSWGQGRGESGQKQVTPDPVTSPQ

SEQ ID NO: 3 - DNA, 2526 bp, Felis catus
atgtcactcccggcggctcacctggtcggcctgcagctctccctctcctgctgctgggctctcagctgccacagcacagagacgtctgccgacttcagcctccctggggattacctcctcgcaggtctgttccctctgcactctgactgtccgggcgtgaggcaccggcccacggtgaccctctgtgacaggcccgacagcttcaacggtcacggctaccacctcttccaggccatgcggtttggcatcgaggagataaacaactccacggccctcctgccgaacgtcaccctgggataccagctgtacgacgtgtgctcggagtctgccaacgtgtatgccacactaaacgtgctctccctgctggggacacatcacgtagagatccgagcagacccttcccactattcgcctgccgccctggctgtcattgggcctgacaccaccaaccacgcagccaccactgcagccctgctgagccccttcctggtgcccctgatcagctacgaggccagcagcgtgacgctcggagtgaagcggcattacccctcgtttctgcgcaccatccccagcgacaagcaccaggtggaggccatggtgctgctgctgcagagcttcgggtgggtctggatctcggtggtcggcagcgacggcgactacgggcagctgggggtgcaggcgctggaggagcaggccacccagcagggcatctgcgttgccttcaaggacatcatccccttctctgcccggccgggcgacgagaggatgcagagcatcatgcaccacctggcccgagcgaggaccaccgttgtggtcgttttctccagcaggcagctggccagggtgttctttgagtcggtggtgctggccaacctgactgccaaggtgtggatcgcctcagaagactgggccatctctagacacatcagcaatgtgcccgggatccagggcattggcacggtgctgggtgtggccatccagcagaggcttgtccctggcctgaaggagtttgaagaggcctatgtccaggcagataagggggcccctgggccttgctccaggacctccgagtgcagcagcaaccagctctgtagagagtgtcgggctttcacggcagagcagatgcccacgctcggggcattctccatgagctctgcttataacgcctaccgggcagtctacgcagtggcccatggcctccaccagctcctgggctgtgcctctggagcctgttccagggaccgagtctacccctggcagcttctggagcagatccgcaaggtgaatttcctcctacacaaggacaccgtgaggtttaatgacaacggggaccctctcagtggctacgacataattgcctgggactggagtggccccaagtggaacttcagggtcattggctcctccatgtggcctccagttcagctggacataaataaaaccaaaatccggtggcacgggaaggacaaccaggtgccaaagtctgtgtgctccagcgactgcctcgaagggcaccagcgagtgatttcgggtttctaccactgttgctttgagtgtgtgccctgtgaggccgggagcttcctcaacaagagcgacctccacagctgccagccttgtgggaaagaaaagtgggcacccgcgggaagtgaaacctgctttccacgcaccgtggtgtttttgacttggcacgagaccatctcttgggtgctgctggcagctaatacgttgctgctgctgctggtgactgggactgctggcctgtttgcctggcacttagacacccctgtggtgaagtccgctgggggccgactgtgcttcttcatgctaggctccctggcagggggcagctgtgggctctacggcttttttggggagcccacgctgcccacatgcttgttgcgccaaagcctccttgccctgggttttgccatcttcctgtcctgcctgaccatccgctccttccaactggtcttcatcttcaagttttctgccaaggtacccaccttctaccgtgcctgggtccaaaaccacggtcctggcctatttgtggtgatcagctcaatggcccagctgctcatctgtctaacttggctggcggtgtggaccccactgcccaccagggagtaccagcgcttccctcagctggtggtgcttgattgcacagaggccaactcaccgggcttcatgttggctttcgcctacaatggcctcctgtccgtcagcgcctttgcctgcagctacctgggcaaggacctgccagagaactacaacgaggccaaatgtgtcacttttagtctgctgctcaacttcgtgtcctggattgccttcttcaccacggccagcgtctaccagggcaagtacttgcccgcggtcaacgtgctggcggcgctgagcagcctgagtggcggcttcagcggttatttcctccccaagtgctacgtgatcctgtgccgcccaaaatttaacagcacacagcacttccaggcctccatccaggagtacacgaggcgctgcggctccacctga

SEQ ID NO: 4 - DNA, 2598 bp, Felis catus
atgcccggcctcgctctcctgggcctcacggctctcctgggcctcacggctctcttggaccacggggagggcgcaacgtcctgcttgtcacagcagctcaggatgcagggggactatgtgctgggtgggctcttccctctgggctctgccgagggtacaggtcttggcgacgggctgcagcccaatgccaccgtgtgcaccaggttctcgtctctgggcctgctctgggcgctggccgtgaagatggcggtggaggagatcaacaacgggtcggccctgctgcccgggctgcacctgggctatgacctctttgacacgtgttcagagcccatggtggccatgaagcccagcctcgtgttcatggccaaagcaggcagctgcagcattgccgcctactgcaattacacacagtaccagccccgcgtgctggccgtcatcgggccccactcgtctgagctcgccctcgtcaccggcaagttcttcagcttcttccttgtgcctcaggtcagctacggcgccagcaccgaccggctgagcaaccgggagatcttcccgtccttcttccgcacggtgcccagcgaccaggtgcaggtggcggccatggtggagctgctggaggagctcggctggaactgggtggcggcggtgggtagtgacgacgagtatggccggcagggcctgagcctcttctccggcctggccagcgccaggggcatctgcatcgcgcatgagggcctggtgccactgccgccaggcagcctgcggctgggcgccctacagggcctgctgcgccaggtgaaccagagcagcgtgcaggtggtggtgctgttctcctccgcccacgcggcccgcaccctcttcagctacagcatccgctgcaagctctcacccaaggtgtgggtggccagcgaggcctggctgacctcagacctggtcatgacgctgcccggcatgcctggggtgggcaccgtgctgggcttcctgcagcagggcgccccgatgccggagttcccatcctacgtgcggacccgcctggccctggccgctgaccctgccttctgcgcctcgctggacgctgaacagccaggcctggaggagcacgtggtggggccacgctgcccccaatgtgaccacgtcacgctagagaacctatctgcggggctgctgcaccaccagaccttcgctgcctacgcggctgtgtatggcgtggcccaagcccttcacaacacactgcgctgcaatgcctcgggctgccccaggcgggagcctgtgcggccctggcagctcctagagaacatgtacaacgtgagcttccgtgctcgcggcctggcactgcagttcgacgccagcgggaacgtgaacgtggattacgacctgaaactgtgggtgtggcaggacccgacgcccgagctgcgcaccgtaggcaccttcaagggccgcctggagctctggcgctctcagatgtgctggcacacgccggggaagcagcagcccgtgtcccagtgctcccggcagtgcaaggaaggccaggtgcgccgcgtgaagggcttccactcttgctgttacaactgcgtggactgcaaggcgggcagttatcagcgcaacccagatgacctcctctgcacccagtgtgaccaggaccagtggtccccagaccggagcacacgctgcttcgcccgcaagcccatgttcctggcatggggggagccagctgtgctgctactgctcgcgctgctggctctggcgctgggcctggcgctggcagccctggggctcttcctctggcactcggacagcccgctggttcaggcctcaggtgggccacgggcctgctttggcctggcttgcctgggcctggtctgcctcagtgtcctcctgttccctggccagccaggccctgccagctgcctggcccagcagccactgttccacctcccactcactggctgcctgagcacgtttttcctgcaagcggccgagatatttgtggggtcggagctgccaccaagctgggctgagaagatgcgtggccgcctgcgggggccctgggcctggctggtggtgctgcttgctatgctggcagaagccgcattgtgtgcctggtacctggtagccttcccgccagaggtggtgacggactggcgggtactgcccacagaggcgctggtgcactgccacgtgcactcctggatcagcttcggcctggtgcatgccactaacgccatgctggccttcctctgcttcctgggcactttcctggtgcagagccggccaggccgctacaatggtgcccgcggcctcacctttgccatgctggcctacttcatcacctggatctcctttgtgcccctctttgccaatgtgcacgtggcctaccagcctgccgtgcagatgggcaccatcctcctctgtgccctgggtatcctagccaccttccacctgcccaagtgctacctgctgctgcagcggccggagctcaacacccctgagttcttcctggaagacaatgccagagcacagggcagcagttgggggcaggggaggggagaatcggggcaaaaacaagtgacacccgatccagtgacctcaccgcagtga